(19) United States
(12) Patent Application Publication
(10) Pub. No.: US 2026/0018302 A1
(43) Pub. Date: Jan. 15, 2026

(54) AI-BASED METHODS AND SYSTEMS FOR PREDICTING DIABETES RISK AND RELATED METABOLIC PARAMETERS

(71) Applicants: Saroj GUPTA, Overland Park,, KS (US); Abhimanyu GUPTA, Overland Park,, KS (US)

(72) Inventors: Saroj GUPTA, Overland Park,, KS (US); Abhimanyu GUPTA, Overland Park,, KS (US)

(73) Assignee: MyDigiRecords, Inc., Kansas City, MO (US)

(21) Appl. No.: 19/263,530

(22) Filed: Jul. 9, 2025

Related U.S. Application Data
(60) Provisional application No. 63/669,704, filed on Jul. 11, 2024.

(51) Int. Cl. G16H 50/30; A61B 5/00; A61B 5/107; A61B 5/16; G06T 7/11

(52) U.S. Cl. CPC G16H 50/30; A61B 5/107; A61B 5/165; A61B 5/4872; G06T 7/11

(57) ABSTRACT

Present disclosure describes techniques for predicting diabetes risk in patients. The techniques include the step of monitoring a plurality of patient-specific characteristics comprising, at least one physiological parameter, one behavioral indicator, and one visual representation of the patient. The method further comprises extracting, using a first artificial intelligence (AI) model, a stress level of the patient based at least on behavioral indicators, historical lifestyle data, and sensor-derived physiological parameters. The method then include extracting, using a second AI model, a body mass index (BMI) or fat distribution patterns based at least on silhouette images and weight of the patient. The method finally includes predicting, using a third AI model, a blood sugar level or diabetes risk score of the patient based on outputs from the first and second AI models and the monitored characteristics.

### BACKGROUND OF THE INVENTION

### Technical Field

The present disclosure relates to artificial intelligence (AI) and machine learning based to system and method for diabetes prediction based on critical medical attributes.

### Description of the Related Art

Diabetes is a metabolic disorder that afflicts tens of millions of people throughout the world. Diabetes results from the inability of the body to properly utilize and metabolize carbohydrates, particularly glucose. Normally, the finely tuned balance between glucose in the blood and glucose in bodily tissue cells is maintained by insulin, a hormone produced by the pancreas which controls, among other things, the transfer of glucose from blood into body tissue cells. Upsetting this balance causes many complications and pathologies including heart disease, coronary and peripheral artery sclerosis, peripheral neuropathies, retinal damage, cataracts, hypertension, coma, and death from hypoglycaemic shock.

Diabetes is one of the chronic diseases that will lead to severe complications in human beings. It occurs when the higher glucose concentration is observed in blood because pancreas does not release sufficient insulin to suppress the additional glucose content. Insulin deficiency causes metabolism disorder which impact the patient's physical and mental health. Early diagnosis and treatment of diabetic is vital to prevent its severity to kidneys, eyes, heart, foots and other organs.

Basically, there are two types of diabetes namely, type-1 diabetes and type-2 diabetes. Type-1 diabetes is severe form of diabetes that requires proper medications and insulin therapy too. It can impact human of any age but generally, teenagers and middle-aged people are more prone to this disorder. However, type-2 diabetics cannot be completely cured but it can be controlled by adopting healthy lifestyle and proper medication if diagnosed at an early stage. Middle to old age people are more likely to have this type of diabetes.

To detect diabetes early, there is a need of an automated system which can consider clinical history as well as physical attributes of the patients for accurate prediction. The physical attributes include blood pressure history, weight, physiological parameters, eating habits, age and lifestyle. Continuous monitoring and analysis of these parameters help to diagnose diabetics so that proper medication and treatment can be extended to the patients. Abnormal increase in glucose in blood level leads to critical diseases such as diabetics retinopathy, diabetic foot ulcer, cardiovascular disorders, diabetic nephropathy, and many others. These diseases may result in loss of eye vision, amputation of foot, kidney dialysis, heart angiography and other criticality if not cured properly. If the sugar level remains high and untreated for a prolonged duration, these diseases may also claim life of the patient.

Recently, machine learning and deep learning algorithms have provided innovative and automatic methodology to diagnose diabetic disorder in human being. DL-based algorithms are fast and accurate in diabetes diagnosis in comparison to conventional prediction methods. For developing robust prediction model, authors have exploited multiple attributes such as pregnancy, plasma glucose, blood pressure, BMI, age, sex, protein profile, dietary examination are few to name. The authors have achieved an accuracy of about 98% using machine learning and deep learning techniques such as logistic regression, naïve Bayes, adaboost, ANN, LSTM and SVM. Out of these models, Adaboost have shown good accuracy in comparison to other models.

However, the existing systems lack in proper identification of sugar level as they do not critical parameters required for accurate sugar level prediction. Thus, there exist a need in the art to provide a technique which overcomes the above-mentioned problems, system and method for diabetes prediction to diagnose diabetic disorder in human being.

For the aforementioned reasons, there exist a need in the art to provide a technique which overcomes the above-mentioned problems and efficiently and effectively detect diabetic disorder using existing capabilities of smart phones and wearable device that are widely available.

### SUMMARY OF THE INVENTION

In one non-limiting embodiment of the present disclosure, an artificial intelligence (AI) based method for predicting diabetes risk in patients is disclosed. The method comprises monitoring a plurality of patient-specific characteristics comprising, at least one physiological parameter, one behavioral indicator, and one visual representation of the patient and extracting, using a first artificial intelligence (AI) model, a stress level of the patient based at least on behavioral indicators, historical lifestyle data, and sensor-derived physiological parameters. The method further comprises extracting, using a second AI model, a body mass index (BMI) or fat distribution patterns based at least on silhouette images and weight of the patient. The method finally includes predicting, using a third AI model, a blood sugar level or diabetes risk score of the patient based on outputs from the first and second AI models and the monitored characteristics.

In another non-limiting embodiment of the present disclosure, the method further comprises generating an alert when the predicted blood sugar level exceeds a predefined threshold.

In another non-limiting embodiment of the present disclosure, the method further comprises adapting the third AI model over time based on longitudinal health data of the patient to increase predictive accuracy, wherein the third AI model comprises a temporal neural network trained on sequential patient records.

In another non-limiting embodiment of the present disclosure, wherein at least one of the first, second, or third AI models is trained or updated via a federated-learning procedure in which model-parameter updates are exchanged with a coordination server while raw patient data remains stored exclusively on the patient's device.

In another non-limiting embodiment of the present disclosure, the extracting the body mass index (BMI) or fat distribution patterns based at least on silhouette images and weight of the patient comprises extracting a plurality of silhouette images from images of the patient, segmenting silhouette images to isolate torso and limb regions, extracting fat distribution patterns associated with diabetes risk, and estimating, using the second AI model, the BMI of the patient at least based on features extracted from silhouette images and the weight of the patient.

In yet another non-limiting embodiment of the present disclosure, the method comprise training the second AI model with a sample set of extracted features of silhouette images and corresponding samples of weight values along with respective BMI.

In yet another non-limiting embodiment of the present disclosure, the stress level is estimated using responses to mood questionnaires, sensor activity logs, and social interaction metrics.

In yet another non-limiting embodiment of the present disclosure, the method further comprise training the first AI model with a sample set of physiological parameters, lifestyle details, medical history, behavioural patterns, and responses to stress-related questions along with corresponding stress levels.

In yet another non-limiting embodiment of the present disclosure, the method further comprise training the third AI model with a sample set of patient-specific characteristics and corresponding blood sugar levels.

In yet another non-limiting embodiment of the present disclosure, the method further comprises generating, for display on a user or clinician interface, an explainability report that identifies (i) relative contribution weights of the monitored physiological parameter, behavioral indicator, and visual representation, and/or (ii) a confidence score, with respect to the predicted blood-sugar level or diabetes-risk score.

In yet another non-limiting embodiment of the present disclosure, a system for predicting diabetes in patients is disclosed. The system comprises a wearable or edge computing device comprising a memory and a processing unit, one or more sensors configured to collect physiological parameters including at least one of heart rate, oxygen level, and/or blood pressure, a camera module configured to capture silhouette images of the patient, The system further comprises a first AI model executable by the processing unit to extract a stress level of the patient based at least on behavioral indicators, historical lifestyle data, and sensor-derived physiological parameters. The system further comprises a second AI model executable by the processing unit to extract body mass index (BMI) or fat distribution patterns based at least on silhouette images and weight of the patient, and a third AI model executable by the processing unit to predict a blood sugar level or diabetes risk score of the patient based on outputs from the first and second AI models and the monitored characteristics.

In yet another non-limiting embodiment of the present disclosure, a non-transitory computer-readable medium is disclosed. The non-transitory computer-readable medium comprises computer-readable instructions that when executed by a processor causes the processor to perform operations of monitoring a plurality of patient-specific characteristics comprising, at least one physiological parameter, one behavioral indicator, and one visual representation of the patient, extracting, using a first artificial intelligence (AI) model, a stress level of the patient based at least on behavioral indicators, historical lifestyle data, and sensor-derived physiological parameters, extracting, using a second AI model, a body mass index (BMI) or fat distribution patterns based at least on silhouette images and weight of the patient, and predicting, using a third AI model, a blood sugar level or diabetes risk score of the patient based on outputs from the first and second AI models and the monitored characteristics.

The foregoing summary is illustrative only and is not intended to be in any way limiting. In addition to the illustrative embodiments, and features described above, further embodiments, and features will become apparent by reference to the drawings and the following detailed description.

### BRIEF DESCRIPTION OF DRAWINGS

The accompanying drawings, which are incorporated in and constitute a part of this disclosure, illustrate exemplary embodiments and, together with the description, serve to explain the disclosed principles. Some embodiments of system and/or methods in accordance with embodiments of the present subject matter are now described, by way of example only, and with reference to the accompanying Figures, in which.

FIG. 1(a) shows an exemplary environment for non-invasively determining diabetes risk score or blood sugar level, in accordance with some embodiments of the present disclosure; and

FIG. 1(b) shows an exemplary methodology for detecting diabetes, in accordance with some aspects of the present disclosure;

FIG. 2 shows a flow diagram for deep-learning model training and deployment for stress estimation, in accordance with some aspects of the present disclosure;

FIG. 3a illustrates an exemplary block diagram for training an AI model for stress level detection, in accordance with some embodiments of the present disclosure;

FIG. 3b shows a block diagram of training an AI model for BMI and fat distribution patterns detection, in accordance with some embodiments of the present disclosure;

FIG. 3c shows a block diagram of training an AI model for blood sugar characterization, in accordance with some embodiments of the present disclosure;

FIG. 4 shows a block diagram of an artificial intelligence (AI) based device for predicting diabetes risk in patients, in accordance with some embodiments of the present disclosure;

FIG. 5 depicts a flowchart illustrating an exemplary artificial intelligence (AI) based method for predicting diabetes risk in patients, in accordance with some embodiments of the present disclosure;

It should be appreciated by those skilled in the art that any block diagrams herein represent conceptual views of the illustrative systems embodying the principles of the present subject matter. Similarly, it will be appreciated that any flowcharts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION OF THE EMBODIMENTS

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiments thereof have been shown by way of example in the drawings and will be described in detail below. It should be understood, however, that these embodiments are not intended to limit the disclosure to the particular form disclosed, but on the contrary, the disclosure is to cover all modifications, equivalents, and alternatives falling within the spirit and the scope of the disclosure.

The terms “comprise(s)”, “comprising”, “include(s)”, or any other variations thereof, are intended to cover a non-exclusive inclusion, such that a setup, device, apparatus, system, or method that comprises a list of components or steps does not include only those components or steps but may include other components or steps not expressly listed or inherent to such setup or device or apparatus or system or method. In other words, one or more elements in a device or system or apparatus proceeded by “comprises . . . a” does not, without more constraints, preclude the existence of other elements or additional elements in the system.

The expressions like “at least one” and “one or more” may be used interchangeably or in combination throughout the description.

In the following detailed description of the embodiments of the disclosure, reference is made to the accompanying drawings that form a part hereof, and in which are shown by way of illustration of specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense. In the following description, well known functions or constructions are not described in detail since they would obscure the description with unnecessary detail.

Accordingly, the term “module” or “model” should be understood to encompass a tangible entity, be that an entity that is physically constructed permanently configured (hardwired) or temporarily configured (programmed) to operate in a certain manner and/or to perform certain operations described herein.

Accordingly, the terms “Artificial intelligence (AI) model” or “AI Model”, “Machine Learning Model” and “ML Model” or “system” should be understood to encompass a tangible entity, be that an entity that is physically constructed permanently configured (hardwired) or temporarily configured (programmed) to operate in a certain manner and/or to perform certain operations described herein.

The terminology “Machine Learning” and “ML” have same meaning and are alternative used throughout the specification.

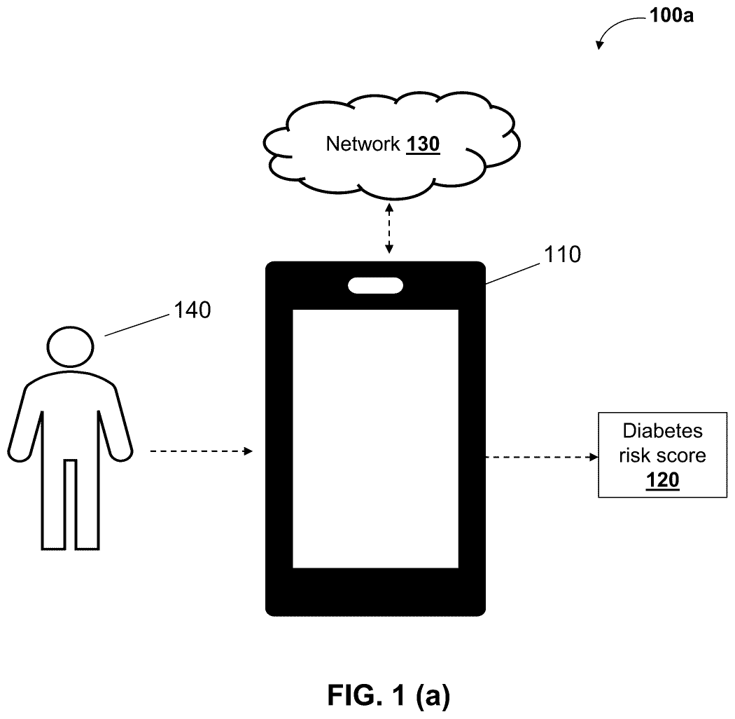

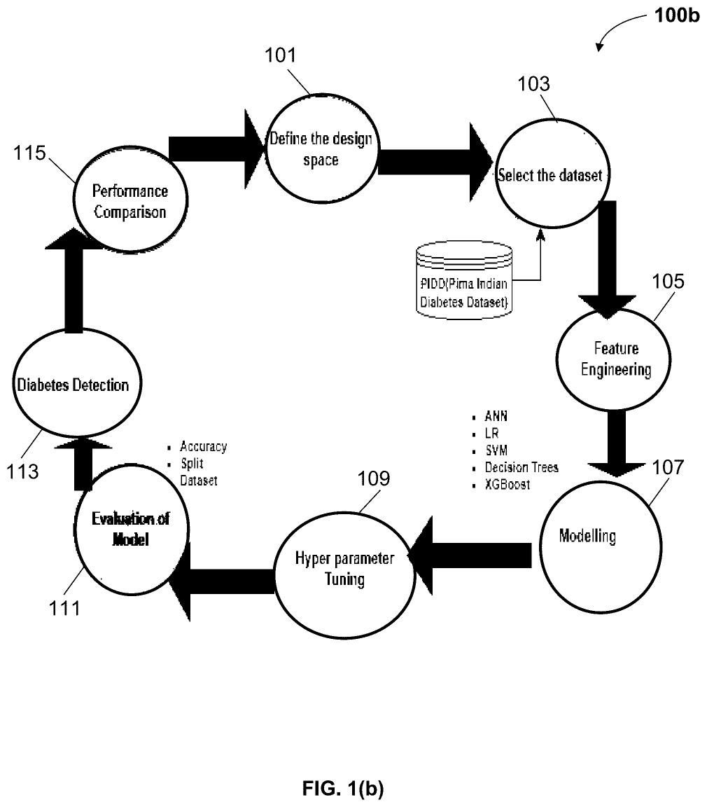

FIG. 1(a) shows an exemplary environment for non-invasively determining diabetes risk score or blood sugar level, in accordance with some embodiments of the present disclosure.

The environment 100 comprises a patient or a subject 101, a user device 110, and a network 130 in communication with each other. The user device 110 may include, for example, a mobile phone, a laptop, a tablet, a desktop computer, and/or the like. However, the user device is not limited to above examples, and may include any other device known to a person skilled in the art.

In an embodiment of the present disclosure, the user device may be communication with smart wearable device worn by the user 140. The smart wearable device may include, but not limited to, smart watches, smart rings, fitness trackers, car phones, smart glasses, and smart clothing, etc. The smart wearable device may measure physiological parameter of the user and provide to the user device 110.

The network 130 may include, for example, the Internet, a private network, public network or other configuration that operates using any one or more known communication protocols such as, for example, transmission control protocol/Internet protocol (TCP/IP). Other examples of the communication protocols include Bluetooth®, BLE®, Wi-Fi, UWB, and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few.

The user device 110 may include a plurality of sensors comprising an image sensor. The user device 110 may be configured to take a plurality of images of a user of the subject or the patient 101. The plurality of images may be then converted to silhouette images. The user device 110 may comprise system for predicting diabetes risk in patients. The user device 110 may be configured to predict, using one or more AI models, blood sugar level or diabetes risk score based on physiological parameter, one behavioral indicator, and one visual representation of the patient. Thus, the user device 101 prevents early diagnosis of diabetes and accurately predict blood sugar level of a person non-invasively.

FIG. 1(b) shows an exemplary methodology 100 for detecting diabetes, in accordance with some aspects of the present disclosure.

At stage 101, a design space is defined. The defining of the design space may comprise developing/training a machine learning (ML) and deep learning models that may be capable of predicting whether a user has diabetes based on certain features. The certain features may be health related attributes associated with the user.

In one non-limiting aspect, the health-related attributes may include parameters such as age, weight, BMI, oxygen level, hypertension, and stress. However, the health-related attributes are not limited to above example and any other health-related attributes known to a person skilled in the art is well within the scope of the present disclosure.

At stage 103, relevant dataset may be selected. The selected dataset may comprise relevant features such as age, BMI, BP, insulin levels, and other health-related metrics. However, the selected health-related metrics are not limited to above example and any other health-related metrics selection is well within the scope of the present disclosure. These health-related metrics may be stored in the memory/cloud server. The selection of the dataset is to ensure the dataset is diverse, representative, and labelled with the target variable indicating diabetes status.

The selection further includes preprocessing of the data. This step shall handle the missing values, outlier detection, and perform feature scaling on the relevant features. Then, the data is processed to understand patterns and relationships. After that the data in the dataset is split into training sets and testing sets.

At stage 105, feature selection is performed. In this step of the method 100, extraction of the meaningful insights from the data is performed at least based on the domain knowledge/domain expertise. In case of missing features, new features may be created or existing ones may be transformed to obtain the main feature. The feature creation and transformation can be performed by using expert administrator having the domain knowledge.

In the next stage 107, modelling of the machine learning and deep learning algorithm/technique is performed. The modelling may include selecting a suitable machine learning and deep learning algorithm for classification tasks. In one non-limiting aspect of the present disclosure, machine learning and deep learning algorithm may be one of, but not limited to Logistic Regression, Decision trees, support vector machine (SVM) a type of supervised learning algorithm, Long Short-Term Memory (LSTM) networks, XGBoost. However, the machine learning model selection are not limited to above examples, and any other machine learning model suitable for a dataset may be selected.

One or more of the above algorithms/techniques may be experimented to find the one that out performs for the specific dataset. Hence, the optimal machine learning and deep learning algorithm/technique is modelled based on the experimentation. However, the experimentation/modelling machine learning and deep learning algorithm are not limited to above example and any other machine learning and deep learning algorithm known to a person skilled in the art is well within the scope of the present disclosure.

At stage 109, training and tuning of the hyperparameters is performed. Training the model with the algorithms and tuning the hyperparameters to improve the overall performance of the model is carried out. In one non-limiting aspect of the present disclosure, the hyperparameters are selected based on their control on the learning process. In particular, the hyperparameters are the values of model parameters that a learning algorithm/technique ends up learning.

In the next stage 111, model is evaluated before the deployment. The evaluation may be performed using the testing dataset obtained during preprocessing. The evaluation of the model may include on the testing dataset using metrics such as accuracy, recall, precision and Flscore. However, the testing/evaluation metrics are not limited to above example and any other testing metrics is well within the scope of the present disclosure. In one non-limiting aspect, the model accuracy may be evaluated using the confusion matrix, which computes the accuracy of the machine learning algorithm in classifying the data into its corresponding labels. In one non-limiting aspect, precision and recall are performance metrics that may be applied to data retrieved from a collection, corpus or sample space.

In the next stage 113, the diabetes detection is performed on the user at least based on the model developed for prediction. In this step, the necessary attributes are retrieved from the user and input to the deep learning model. The deep learning model predicts the glucose level of the patient at least based on the above-mentioned training of the deep learning model.

In one non-limiting aspect, at stage 115, the model performance may be compared with actual value of the glucose level to retrain the deep learning model and improve the performance of the model. In one non-limiting aspect, the actual value of the glucose level may also be used as a means to calibrate the model.

Thus, the method facilitates accurate prediction of sugar level in the user and provide early diagnosis of diabetes that helps in fast and accurate treatment of the disease to prevent its defects at later stages.

In an aspect of the present disclosure, stress or hypertension and BMI may be the critical parameters for prediction of diabetes or glucose level of patient because of the following reason:
Stress is one of the prevalent mental health issues affecting people's lives. It can also be one of the sources for many other critical medical diseases namely, cardiovascular disorder, diabetes, depression, and many other mental health problems. Stress is a psychological disorder that can be predicted in a human by analyzing multiple parameters such as its physical, behavioral and biometric changes. Early, accurate and faster prediction of stress in a person is foremost necessary to prevent the serious health issue and save lives.

In this fastest growing and modest world, people are struggling to perform better and achieve success. The substantial factors namely, lifestyle, social media, work pressure, and business competitions are impacting person's mental health. The physical changes such as increase heart rate, variations in skin conductance and increase in cortisol levels occur in human body which severely impacting the body organs. Apart from physical changes, stress may also lead to behavioral variations in human body. Aggressiveness in speech, changes in facial expressions and restless movement patterns are few behavioral indictors that can be visualize in a person suffering from stress.

Stress influences the mental and emotional state of a person which in term impact its biopsychological attributes. Stress not only impact the internal stimuli but also affect externally which require subjective and objective measurements. Subjective measurement of stress can be done by asking question. The corresponding answers can be evaluated to determine the stress severity. However, this method is not very reliable, and chance of false outcome is relatively high as it is highly dependent on the mood of the person writing answers. Conceptually, stress can be computed accurately by continuously monitoring the person's mental health. It has been widely accepted that it is necessary to monitor the person's vital such as its physical, behavioral and other parameters to measure stress. There is a requirement of method which can measure stress in a person by accurately and effectively.

Stress is a psychological condition that may lead to depression, anxiety, suicidal tendencies and many other metabolism disorders. The person under stress has no control over its activity and behavior. In the initial stages, stress prediction is not an easy task, but its impact on regular activities is highly severe. Hence, it is vital to determine the stress in human in preliminary stages to prevent the propagation of its ill effects.

With the emergence in technology, mobile based applications, and wearable devices are equipped with sensors that can monitor person's mental health effectively. These devices and application can read the biological signals that includes cardiac and brain waves monitoring to predict the person's mental health and mood. These applications collect data that can also be analyzed by AI to predict the symptoms of stress more accurately and efficiently.

In an exemplary aspect, there are two ways to measure stress namely using manual, and AI-based methods. AI-based methods are faster, accurate and cheap in comparison to manual methods. Manual evaluation of stress requires continuous monitoring of person's behavior, lifestyle, clinical history, and other parameters. For which, one must visit doctor either psychologist or psychiatrists to get the solutions.

However, people are not only reluctant but also unaware about their mental illness. Physical visits and discussions are also not possible as people are neither want to disclose their identity nor have acceptance for their mental disorder. In addition, manual methods are not able to analyze one's social media life to predict the stress accurately. Hence, manual methods can be equipped to AI-based prediction methods to get accurate, and trustworthy outcomes.

In recent years, AI-based stress prediction algorithms have gain momentum due to benefits and advantages over manual methods. AI-based methods utilized either machine learning algorithms or deep learning algorithms to predict stress. Based on the severity, these algorithms can also categorize stress levels as normal, low, moderate, and high. Random forest, k-nearest neighbor, support vector machine are few popular machine learning algorithms who have shown efficient and accurate results. However, deep neural networks (DNN) such as artificial neural network (ANN), convolutional neural network (CNN), Recurrent neural network (RNN) can also categorize stress level measurement into multiple classes.

DL-based stress prediction algorithms require multiple features for efficient and accurate results. There are cognitive, emotional, behavioral, contextual, psychological, environmental, and biographical ways contributing to stress in human. In order to analyze this, sleeping patterns, cardiac rate, emotion level, speech rate, eye movement, and social interaction are few paramount parameters that play vital role in stress level estimation. Once model is trained on these parameters, it is able predict stress efficiently.

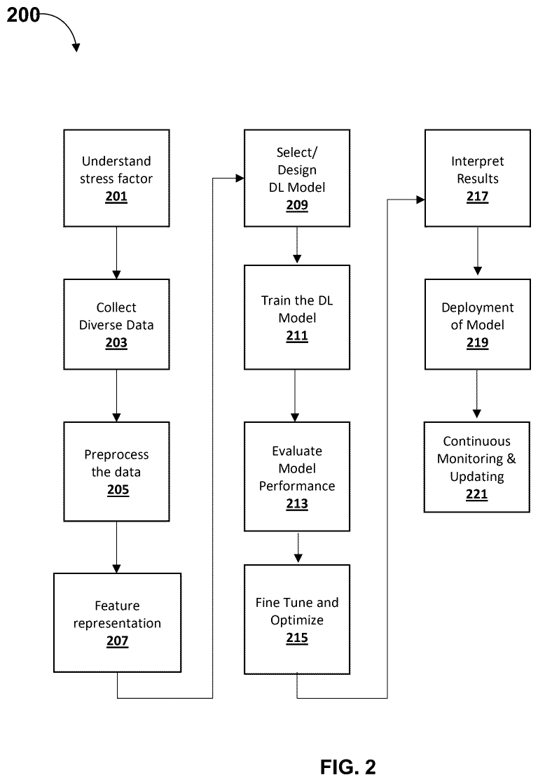

FIG. 2 shows a flow diagram 200 for deep-learning model training and deployment for stress estimation, in accordance with some aspects of the present disclosure.

At step 201, the stress factors are understood. Firstly, the method identifies and understands the various factors contributing to stress. This could include physiological indicators like heart rate, behavioural patterns, and self-reported information. However, the factors contributing to stress is not limited to above example and any other physiological indicator contributing to stress is well with the scope of the present disclosure.

At step 203, collection of diverse data may be performed. The collection includes gathering a diverse dataset that incorporates information on stress-related factors. This could involve physiological measurements, lifestyle details, and responses to stress-related questions. In one no-limiting aspect, other ways of obtaining the diverse data set may also be implemented. In one non-limiting aspect, a questionnaire for determining the stress level of user may be framed and presented to the user at regular interval for determining the psychological condition of the user.

At step 205, the received diverse data is pre-processed. The preprocessing includes clean and prepare the data for analysis. This may involve handle of missing values, standardize numerical features, and encode categorical variables to ensure the data is suitable for deep learning models. In one non-limiting aspect of the present disclosure, handling missing values, standardize numerical features, and encode categorical variables may be carried out using suitable techniques known to a person skilled in the art.

At step 207, feature representation may be performed. The feature representation may include representing the stress-related features in a way that deep learning models can understand. This may involve techniques like normalization or embedding for categorical variables or any other technique known to a person skilled in the art.

At step 209, a Deep Learning Model is selected and modelled/designed for stress estimation. The Deep Learning Model used for estimation may be one of Recurrent Neural Networks (RNNs) or Long Short-Term Memory (LSTM) networks whichever is effective for handling sequential data, while feedforward neural networks might work well for tabular data. However, the Deep Learning Model selection is not limited to above example, and any other model known to a person skilled in the art is well within the scope of the present disclosure.

At step 211, the selected Deep Learning Model is trained. In the step of training the selected deep learning model is trained using the pre-processed data. This may also involve adjustment of the hyperparameters, such as learning rate and batch size, to optimize the model's performance.

At step 213, evaluation/testing of the Deep Learning Model to check the performance of the model. In an aspect, the evaluation/testing may be performed based on the pre-processed data. Assessment of the model's performance may be performed using a separate set of data, which is not used during training. Common evaluation metrics for classification tasks include accuracy, precision, recall, and F1-score. Or any other metrics may be used to evaluate the performance of the model.

At step 215, fine-tune and optimization of the Deep Learning Model. The deviation observed during the evaluation may be used as feedback to retrain the Deep Learning Model. In other words, the evaluation results may be used to fine-tune your model. Experiment with different architectures, adjust hyperparameters, and consider techniques like dropout to prevent overfitting.

At step 217, the results may be interpreted. If interpretability is essential, methods are explored to interpret the deep learning model's predictions. Visualization techniques or attention mechanisms in models like LSTMs can provide insights into what the model focuses on when predicting stress.

At step 219, the trained deep learning model may be deployed after the fine tune is completed. Once satisfied with the model's performance, deploy it in a real-world setting for stress prediction.

At step 221, continuous monitoring and updating of the deep learning model is performed. This involves regularly monitoring the model's performance in real-time and update it as needed. This ensures the model stays effective as new data becomes available.

Thus, the above-mentioned technique facilitates efficient deep learning model training and accurate stress level estimation.

The above procedure of flow diagram 200 may also be used to predict any the other critical parameter required for estimating the stress level of the user.

For example, another critical parameter required for estimating the stress level of the user may be body mass index (BMI). The BMI of the use may be determined as follows:

BMI of a person may be calculated by exploiting silhouette images using deep learning algorithms. Silhouette images are extracted from the original images of the user. The silhouette images contain binary information, and require less computations for processing. Single full frontal colour images of the users are processed for generating the silhouette images.

Initially, the images are outlined black to mark the user in the image manually. These manually marked images are non-uniform and noisy. These images vary in markings and contain artefacts in the form of tape markings. Also, the background noise and environmental variations such as illumination variations, blurriness and scale variations are introduced in the image due to image capturing device. In order to reduce the impact of noise, all images are converted to grayscale in PNG format with uniform fixed resolution and background is cropped considering the aspect ratio.

Thereafter, features are extracted from the processed image. Primarily, two features namely, Gabor filter and Histogram of Oriented Gradients (HOG) are extracted. Gabor filter is initialized with wide range of parameters to optimize the feature generation process. The feature vector generated using Gabor filter and HOG are utilized for training the machine learning (ML) model.

Further, multiple ML model including a combination of random forest, XGBoost, SVM for selecting high-level features. Randomised search along with cross validation are used to optimize the network parameters to improve its efficiency. Finally, the modified VGGNet, CNN is used for processing the selected features to obtain the results.

The model architecture contains two filter layers, 4×8 filter, ReLU activation, BN and two dense layer as single output layer. Lastly, the flatten layer comprises of fully connected CNN layer, ReLU, BN and dropout followed by fully connected layer and ReLU again. Mean squared error as loss function, GridSearch of parameter optimal selection, and Adam algorithm as optimizer to obtain the normalized BMI from input images.

However, the BMI estimation/calculation is not limited to above exemplified technique and any other technique known to a person skilled in the art is well within the scope of the present disclosure.

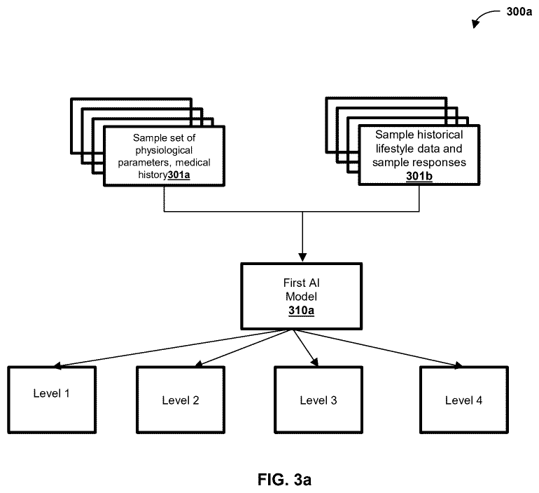

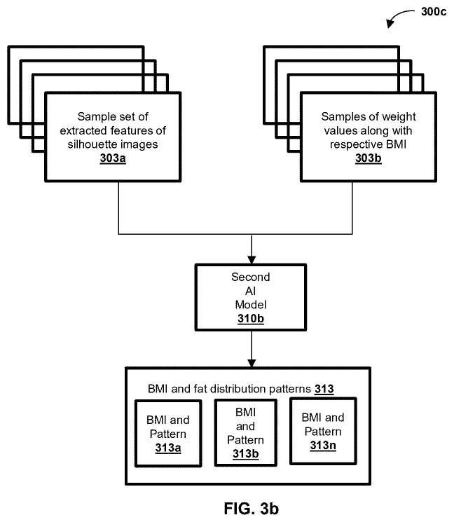

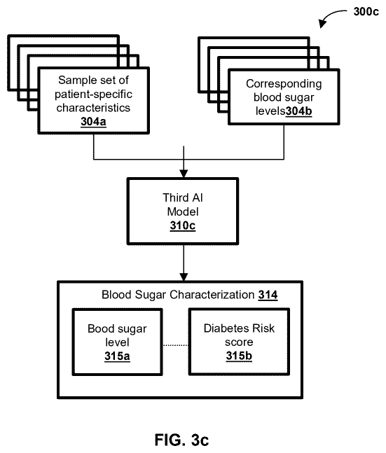

FIG. 3a illustrates an exemplary block diagram for training an AI model for stress level detection, in accordance with some embodiments of the present disclosure.

In an aspect of the present disclosure, an AI model or a first AI model 310a may be trained with a sample set of physiological parameters and medical history 301a, and sample historical lifestyle data and sample responses of questionnaire 301b along with corresponding stress level values.

The first AI model 310a may be configured to receive a plurality of sample set of physiological parameters and medical history 301a, and sample historical lifestyle data and sample responses 301b and stress level. The AI-ML model 310 may be configured to classify the physiological parameters, medical history, historical lifestyle data, and sample responses of questionnaire to different stress levels (level-1, level-2, level 3, level 4).

In an embodiment of the present disclosure, the first AI model 310a may be configured to estimated stress level based on real-time responses to mood questionnaires, sensor activity logs, and social interaction metrics. Thus, the first AI model 310a facilitates in accurate detection of stress level of a user.

FIG. 3b shows a block diagram of training an AI model for BMI and fat distribution patterns detection, in accordance with some embodiments of the present disclosure.

In an aspect of the present disclosure, BMI and fat distribution patterns may be determined based on training of an AI model or a second AI model 310b discussed in reference with FIG. 3b. The second AI model 310b may be trained with a plurality of sample set of extracted features of silhouette images 303a and samples of weight values along with respective BMI 303b.

The second AI model 310b may also be trained for feature extraction such as detection of size, shape, and contour of silhouette images 303a and for classification of different size, shape, and contour with BMI and fat distribution patterns 313. The BMI and fat distribution patterns 313 may comprise BMI and Pattern 313a, 313b, . . . 313n.

In one non-limiting aspect, the trained second AI model 310b may may be a deep neural network or convolution neural network model. Further, the feature detection technique may include one of: Harris Corner Detection, Shi-Tomasi Corner Detection, Canny Edge Detection, Blob Detection, Scale-Invariant Feature Transform (SIFT), etc. However, the BMI and fat distribution patterns detection technique is not limited to above example and any other technique known to a person skilled in the art is well within the scope of present disclosure.

Once the second AI model 310b is trained, the second AI model 310b may be used to accurately determine BMI and fat distribution patterns of different users/patients.

FIG. 3c shows a block diagram of training an AI model for blood sugar characterization, in accordance with some embodiments of the present disclosure.

In an aspect of the present disclosure, an AI model or a third AI model 310c may be provided a plurality of sample set of patient-specific characteristics 304a and corresponding blood sugar levels 304b. The AI-ML model 310 is trained for estimation of blood sugar levels and diabetes risk score based on patient-specific characteristics. Thus, the AI-ML model 310 may classify each patient-specific characteristics with respective blood sugar levels 315a and diabetes risk score 315b.

In an embodiment, the third AI model 310c may rely on output of the first AI model 310a and the second AI model 310b for providing the blood sugar levels and diabetes risk score. In one non-limiting embodiment, the blood sugar levels and diabetes risk score may be predicted using a single model trained with functionalities of the first AI model 310a, the second AI model 310b, and the third AI model 310c.

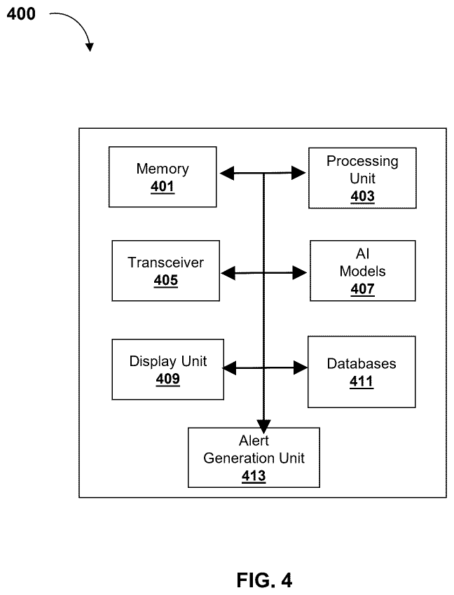

FIG. 4 shows a block diagram of an artificial intelligence (AI) based device for predicting diabetes risk in patients, in accordance with some embodiments of the present disclosure.

In an aspect of the present disclosure, the diabetes detecting system 400 may comprise a memory 401, a processing unit 403, a transceiver 405, AI models/modules 407, display unit 409, databases 411, and alert generation unit 413 communicatively coupled with each other. The diabetes detecting system 400 may be in communication with the cloud/server for retrieving data stored at the cloud/server.

In an embodiment of the present disclosure, the AI models/modules 407 may comprise the first AI model 310a, the second AI model 310b, and the third AI model 310c, as discussed in above embodiments. In one non-limiting embodiment, the diabetes detecting system 400 may comprise a wearable or edge computing device comprising the memory 401 and the processing unit 403.

In addition, the diabetes detecting system 400 may also comprise one or more sensors configured to collect physiological parameters including at least one of heart rate, oxygen level, and/or blood pressure and a camera module configured to capture silhouette images of the patient. In one non-limiting embodiment, already stored recent images of the patient may be taken and converted to silhouette images.

In one aspect of the present disclosure, the memory 401 may be configured to store the diverse data of user/patient. In one non-limiting aspect, the processing unit 403 may be configured to retrieve the diverse data of user/patient from the cloud and store in the memory 401. The memory 401 may also be configured to store medical reports comprising mental status of the patient, which gets updated at regular interval as the state of patient changes with time.

The processing unit 303 may be configured to extract, using the first AI model, a stress level of the patient based at least on behavioral indicators, historical lifestyle data, and sensor-derived physiological parameters. The stress level may also be estimated using responses to mood questionnaires, sensor activity logs, and social interaction metrics.

In an embodiment, the processing unit 403 may be configured to train the first AI model with a sample set of physiological parameters, lifestyle details, medical history, behavioural patterns, and responses to stress-related questions along with corresponding stress levels. This training may be useful in above mentioned stress level of the patient.

In an aspect, the processing unit 303 may be configured to detect user condition from mental and emotional status of a user. The mental and emotional status may be detected using proper diagnostic technique known to a person skilled in the art. In one non-limiting aspect, the user/patient behavior is continuously monitored based his/her interaction on social networking platforms and activities user/patient. However, the user condition detection is not limited to above example and any other technique used for detecting user condition is well within the scope of present disclosure.

The processing unit 403 also be configured to extract, using the second AI model, body mass index (BMI) or fat distribution patterns based at least on silhouette images and weight of the patient. The extraction may require processing unit 403 to extract a plurality of silhouette images from images of the patient, segment silhouette images to isolate torso and limb regions, extract fat distribution patterns associated with diabetes risk, and estimate, using the second AI model, the BMI of the patient at least based on features extracted from silhouette images and the weight of the patient.

In an embodiment, the second AI model may be trained by the processing unit 403. The processing unit 403 may be configured to train the second AI model with a sample set of extracted features of silhouette images and corresponding samples of weight values along with respective BMI, as discussed in explanation of FIG. 3(b).

The processing unit 403 may be then configured to predict, using the third AI model, a blood sugar level or diabetes risk score of the patient based on outputs from the first and second AI models and the monitored characteristics. In an aspect, the processing unit 403 may be configured to train the third AI model with a sample set of patient-specific characteristics and corresponding blood sugar levels for such prediction.

In an aspect, the databases 411 may store the domain knowledge or domain expertise or clinical knowledge. However, the database 411 may also support with possible data/information require for diabetes level estimation.

In an aspect of the present disclosure, the processing unit 403 may be configured to generate an alert when the predicted blood sugar level exceeds a predefined threshold. The processing unit 403 may be configured to determine diabetes risk score of the patient at least based on the predicted blood sugar level.

In an aspect of the present disclosure, the processing unit 403 may be configured to adapt the third AI model over time based on longitudinal health data of the patient to increase predictive accuracy, wherein the third AI model comprises a temporal neural network trained on sequential patient records. However, the adaptation is not restricted to third AI model and adaption of the first and second AI model based on longitudinal health data of the patient to increase predictive accuracy, is well within the scope of present disclosure.

In one non-limiting aspect, at least one of the first, second, or third AI models is trained or updated via a federated-learning procedure in which model-parameter updates are exchanged with a coordination server while raw patient data remains stored exclusively on the patient's device.

In an aspect of the present disclosure, the processing unit 403 may be configured to generate, for display on a user or clinician interface, an explainability report that identifies (i) relative contribution weights of the monitored physiological parameter, behavioral indicator, and visual representation, and/or (ii) a confidence score, with respect to the predicted blood-sugar level or diabetes-risk score.

Thus, the system 400 consider the salient features extracted from the persons in real-time to accurately predict the diabetes level in person and early diagnosis of diabetes will help in the fast and accurate treatment of the disease to prevent its defects at later stages.

It may be noted that, in some embodiments, the system 400 may include more or fewer components than those depicted herein. The various components of the system 400 may be implemented using hardware, software, firmware or any combinations thereof. Further, the various components of the system 400 may be operably coupled with each other. More specifically, various components of the system 400 may be capable of communicating with each other using communication channel media (such as buses, interconnects, etc.).

In one embodiment, the processing unit 403 may be embodied as a multi-core processor, a single core processor, or a combination of one or more multi-core processors and one or more single core processors. For example, the processing unit 403 may be embodied as one or more of various processing devices, such as a coprocessor, a microprocessor, a controller, a digital signal processor (DSP), a processing circuitry with or without an accompanying DSP, or various other processing devices including, a microcontroller unit (MCU), a hardware accelerator, a special-purpose computer chip, or the like.

In one embodiment, the memory 401 is capable of storing machine executable instructions, referred to herein as instructions. The instructions may be written in Python language. In an embodiment, the processing unit 303 is embodied as an executor of software instructions. As such, the processing unit 303 is capable of executing the instructions stored in the memory 301 to perform one or more operations described herein.

The memory 401 can be any type of storage accessible to the processing unit 403 to perform respective functionalities. For example, the memory 401 may include one or more volatile or non-volatile memories, or a combination thereof. For example, the memory 401 may be embodied as semiconductor memories, such as flash memory, mask ROM, PROM (programmable ROM), EPROM (erasable PROM), RAM (random access memory), etc. and the like.

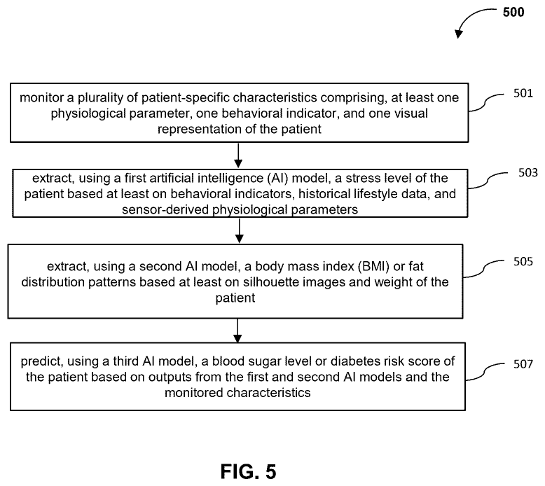

FIG. 5 depicts a flowchart illustrating an exemplary artificial intelligence (AI) based method for predicting diabetes risk in patients, in accordance with some embodiments of the present disclosure.

At step 501, the method 500 discloses monitoring a plurality of patient-specific characteristics comprising, at least one physiological parameter, one behavioral indicator, and one visual representation of the patient. The physiological parameter may be monitored using one or more sensors of a wearable or user device.

At step 503, the method 500 discloses extracting, using a first artificial intelligence (AI) model, a stress level of the patient based at least on behavioral indicators, historical lifestyle data, and sensor-derived physiological parameters. The stress level is estimated using responses to mood questionnaires, sensor activity logs, and social interaction metrics.

In an aspect of the present disclosure, the method 500 discloses training the first AI model with a sample set of physiological parameters, lifestyle details, medical history, behavioral patterns, and responses to stress-related questions along with corresponding stress levels, as explained in reference to FIG. 3(a).

At step 505, the method 500 discloses extracting, using a second AI model, a body mass index (BMI) or fat distribution patterns based at least on silhouette images and weight of the patient. The extraction of the body mass index (BMI) or fat distribution patterns based at least on silhouette images and weight of the patient comprises extracting a plurality of silhouette images from images of the patient, segmenting silhouette images to isolate torso and limb regions, extracting fat distribution patterns associated with diabetes risk, and estimating, using the second AI model, the BMI of the patient at least based on features extracted from silhouette images and the weight of the patient.

In an embodiment, the method 500 discloses training the second AI model with a sample set of extracted features of silhouette images and corresponding samples of weight values along with respective BMI.

At step 507, the method 500 discloses predicting, using a third AI model, a blood sugar level or diabetes risk score of the patient based on outputs from the first and second AI models and the monitored characteristics. Further, the method discloses generating an alert when the predicted blood sugar level exceeds a predefined threshold.

In an embodiment, the method 500 discloses training the third AI model with a sample set of patient-specific characteristics and corresponding blood sugar levels. Further, the method 500 discloses adapting the third AI model over time based on longitudinal health data of the patient to increase predictive accuracy, wherein the third AI model comprises a temporal neural network trained on sequential patient records. However, the adaptation is not restricted to third AI model and adaption of the first and second AI model based on longitudinal health data of the patient to increase predictive accuracy, is well within the scope of present disclosure.

In an embodiment, at least one of the first, second, or third AI models is trained or updated via a federated-learning procedure in which model-parameter updates are exchanged with a coordination server while raw patient data remains stored exclusively on the patient's device.

In an embodiment, the method 500 discloses generating, for display on a user or clinician interface of a user, an explainability report that identifies (i) relative contribution weights of the monitored physiological parameter, behavioral indicator, and visual representation, and/or (ii) a confidence score, with respect to the predicted blood-sugar level or diabetes-risk score.

Thus, the method 500 consider the salient features extracted from the persons in real-time to accurately predict the diabetes level in person and early diagnosis of diabetes will help in the fast and accurate treatment of the disease to prevent its defects at later stages.

In another non-limiting embodiment of the present disclosure, the steps of method 500 may be performed in an order different from the order described above.

It is to be understood that not necessarily all objectives or advantages may be achieved in accordance with any particular embodiment described herein. Thus, for example, those skilled in the art will appreciate that certain embodiments may be configured to operate in a manner that achieves or optimizes one advantage or group of advantages as taught herein without necessarily achieving other objects or advantages as may be taught or suggested herein.

In a non-limiting embodiment of the present disclosure, one or more non-transitory computer-readable media may be utilized for implementing the embodiments consistent with the present disclosure. A computer-readable media refers to any type of physical memory (such as the memory) on which information or data readable by a processor may be stored. Thus, a computer-readable media may store one or more instructions for execution by the at least one processor, including instructions for causing the at least one processor to perform steps or stages consistent with the embodiments described herein. The term “computer-readable media” should be understood to include tangible items and exclude carrier waves and transient signals. By way of example, and not limitation, such computer-readable media can comprise Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

Thus, certain aspects may comprise a computer program product for performing the operations presented herein. For example, such a computer program product may comprise a computer readable media having instructions stored (and/or encoded) thereon, the instructions being executable by one or more processors to perform the operations described herein. For certain aspects, the computer program product may include packaging material.

The terms “an embodiment”, “embodiment”, “embodiments”, “the embodiment”, “the embodiments”, “one or more embodiments”, “some embodiments”, and “one embodiment”, “other embodiment”, “yet another embodiment”. “non-limiting embodiment” mean “one or more (but not all) embodiments of the disclosure(s)” unless expressly specified otherwise.

The various exemplary logical blocks and modules described in connection with the embodiments disclosed herein can be implemented or executed by a machine such as a processor. The processor may be a microprocessor, but alternatively, the processor may be a controller, a microcontroller, or a state machine, or a combination thereof. The processor can include an electrical circuit configured to process computer executable instructions. In another embodiment, the processor includes an application specific integrated circuit (ASIC), a field programmable gate array (FPGA), or other programmable device that performs logical operations without processing computer executable instructions. The processor can also be implemented as a combination of computing devices, e.g., a combination of a digital signal processor (DSP) and a microprocessor, a plurality of microprocessors, one or more microprocessors in conjunction with a DSP core, or any other such configuration. Although described herein primarily with respect to digital technology, the processor may also include primarily analog components. For example, some or all of the signal processing algorithms described herein may be implemented by analog circuitry or mixed analog and digital circuitry. A computing environment may include any type of computer system, including, but not limited to, a computer system that is based on a microprocessor, mainframe computer, a digital signal processor, a portable computing device, a device controller, or a computing engine within the device.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

A description of an embodiment with several components in communication with each other does not imply that all such components are required. On the contrary, a variety of optional components are described to illustrate the wide variety of possible embodiments of the disclosed methods and systems.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based here on. Accordingly, the embodiments of the present disclosure are intended to be illustrative, but not limiting, of the scope of the disclosure, which is set forth in the appended claims.

What is claimed is:

1. A method for predicting diabetes risk in patients, the method comprising:
monitoring a plurality of patient-specific characteristics comprising, at least one physiological parameter, one behavioral indicator, and one visual representation of the patient;
extracting, using a first artificial intelligence (AI) model, a stress level of the patient based at least on behavioral indicators, historical lifestyle data, and sensor-derived physiological parameters;
extracting, using a second AI model, a body mass index (BMI) or fat distribution patterns based at least on silhouette images and weight of the patient; and
predicting, using a third AI model, a blood sugar level or diabetes risk score of the patient based on outputs from the first and second AI models and the monitored characteristics.

2. The method of claim 1, further comprising:
generating an alert when the predicted blood sugar level exceeds a predefined threshold.

3. The method of claim 1, further comprising:
adapting the third AI model over time based on longitudinal health data of the patient to increase predictive accuracy, wherein the third AI model comprises a temporal neural network trained on sequential patient records.

4. The method of claim 1, further comprising generating, for display on a user or clinician interface, an explainability report that identifies (i) relative contribution weights of the monitored physiological parameter, behavioral indicator, and visual representation, and/or (ii) a confidence score, with respect to the predicted blood-sugar level or diabetes-risk score.

5. The method of claim 1, wherein extracting the body mass index (BMI) or fat distribution patterns based at least on silhouette images and weight of the patient comprises:
extracting a plurality of silhouette images from images of the patient;
segmenting silhouette images to isolate torso and limb regions;
extracting fat distribution patterns associated with diabetes risk; and
estimating, using the second AI model, the BMI of the patient at least based on features extracted from silhouette images and the weight of the patient.

6. The method of claim 1, further comprising:
training the second AI model with a sample set of extracted features of silhouette images and corresponding samples of weight values along with respective BMI.

7. The method of claim 1, wherein the stress level is estimated using responses to mood questionnaires, sensor activity logs, and social interaction metrics.

8. The method of claim 1, further comprising:
training the first AI model with a sample set of physiological parameters, lifestyle details, medical history, behavioural patterns, and responses to stress-related questions along with corresponding stress levels.

9. The method of claim 1, further comprising:
training the third AI model with a sample set of patient-specific characteristics and corresponding blood sugar levels.

10. The method of claim 1, wherein at least one of the first, second, or third AI models is trained or updated via a federated-learning procedure in which model-parameter updates are exchanged with a coordination server while raw patient data remains stored exclusively on the patient's device.

11. A system for predicting diabetes in patients, the system comprising:
a wearable or edge computing device comprising a memory and a processing unit;
one or more sensors configured to collect physiological parameters including at least one of heart rate, oxygen level, and/or blood pressure;
a camera module configured to capture silhouette images of the patient;
a first AI model executable by the processing unit to extract a stress level of the patient based at least on behavioral indicators, historical lifestyle data, and sensor-derived physiological parameters;
a second AI model executable by the processing unit to extract body mass index (BMI) or fat distribution patterns based at least on silhouette images and weight of the patient; and
a third AI model executable by the processing unit to predict a blood sugar level or diabetes risk score of the patient based on outputs from the first and second AI models and the monitored characteristics.

12. The system of claim 11, wherein the processing unit is further configured to:
generate an alert when the predicted blood sugar level exceeds a predefined threshold.

13. The system of claim 11, wherein the processing unit is further configured to:
adapt the third AI model over time based on longitudinal health data of the patient to increase predictive accuracy, wherein the third AI model comprises a temporal neural network trained on sequential patient records.

14. The system of claim 11, wherein the processing unit is further configured to:
generate, for display on a user or clinician interface, an explainability report that identifies (i) relative contribution weights of the monitored physiological parameter, behavioral indicator, and visual representation, and/or (ii) a confidence score, with respect to the predicted blood-sugar level or diabetes-risk score.

15. The system of claim 11, wherein to extract the body mass index (BMI) or fat distribution patterns based at least on silhouette images and weight of the patient, the processing unit is configured to:
extract a plurality of silhouette images from images of the patient;
segment silhouette images to isolate torso and limb regions;
extract fat distribution patterns associated with diabetes risk; and
estimate, using the second AI model, the BMI of the patient at least based on features extracted from silhouette images and the weight of the patient.

16. The system of claim 11, wherein the processing unit is further configured to:
train the second AI model with a sample set of extracted features of silhouette images and corresponding samples of weight values along with respective BMI.

17. The system of claim 11, wherein the processing unit is further configured to:
train the first AI model with a sample set of physiological parameters, lifestyle details, medical history, behavioural patterns, and responses to stress-related questions along with corresponding stress levels.

18. The system of claim 11, wherein the processing unit is further configured to:
train the third AI model with a sample set of patient-specific characteristics and corresponding blood sugar levels.

19. The system of claim 11, wherein at least one of the first, second, or third AI models is trained or updated via a federated-learning procedure in which model-parameter updates are exchanged with a coordination server while raw patient data remains stored exclusively on the patient's device.

20. A non-transitory computer-readable medium having computer-readable instructions that when executed by a processor causes the processor to perform operations of:
monitoring a plurality of patient-specific characteristics comprising, at least one physiological parameter, one behavioral indicator, and one visual representation of the patient;
extracting, using a first artificial intelligence (AI) model, a stress level of the patient based at least on behavioral indicators, historical lifestyle data, and sensor-derived physiological parameters;
extracting, using a second AI model, a body mass index (BMI) or fat distribution patterns based at least on silhouette images and weight of the patient; and
predicting, using a third AI model, a blood sugar level or diabetes risk score of the patient based on outputs from the first and second AI models and the monitored characteristics.
